(19) United States
(12) Patent Application Publication
(10) Pub. No.: US 2026/0283516 A1
(43) Pub. Date: Sep. 24, 2026

(54) Single Port Collection System for Blood Collection From an A-Line, PIC, or Catheter

(71) Applicant: Becton, Dickinson and Company, Franklin Lakes, NJ (US)

(72) Inventor: Anthony V. Torris, Mercer Island, WA (US)

(21) Appl. No.: 19/473,510

(22) PCT Filed: Apr. 9, 2024
(86) PCT No.: PCT/US2024/023655

§ 371 Date: Oct. 8, 2025

Related U.S. Application Data
(60) Provisional application No. 63/458,245, filed on Apr. 10, 2023.

(51) Int. Cl. A61B 5/15; A61B 5/153; A61B 50/33

(52) U.S. Cl. CPC A61B 5/150992; A61B 5/153; A61B 50/33

(57) ABSTRACT

A blood collection system configured for connection to a tube set providing vascular access to a patient, the system including a discard syringe configured to collect a waste blood sample therein, the discard syringe including a primary connector portion and a secondary port, wherein the secondary port is in fluid communication with the primary connector portion. The system also includes a collection syringe configured to collect a testing blood sample therein, wherein the collection syringe includes a collection syringe connector portion configured to be couplable with the secondary port of the discard syringe.

### CROSS-REFERENCE TO RELATED APPLICATION

The present application claims priority to U.S. Provisional Application No. 63/458,245 entitled “Single Port Collection System for Blood Collection from an A-line, PIC, or Catheter” filed Apr. 10, 2023, the entire disclosure of which is hereby incorporated by reference.

### BACKGROUND OF THE INVENTION

### Field of the Disclosure

The present disclosure relates generally to blood collection devices. More particularly, the present disclosure relates to single port collection system for blood collection from an arterial line (A-line), a peripherally-inserted central catheter (PICC), or other catheter.

### Description of the Related Art

Blood sampling is a common health care procedure involving the withdrawal of at least a drop of blood from a patient. Blood samples are commonly taken from hospitalized, homecare, and emergency room patients either by finger stick, heel stick, or venipuncture. Blood samples may also be taken from patients by venous or arterial lines. Once collected, blood samples may be analyzed to obtain medically useful information including chemical composition, hematology, and coagulation, for example.

For blood draws using existing vascular access, the venous or arterial lines may be coupled to, e.g., a stopcock medical connector having a single luer connection port extending therefrom for connection to various blood collection devices. However, typical blood collection procedures using such a connector involve multiple interactions with the luer connection port, which may increase the risk of contamination and/or slow the workflow of the healthcare professional. For example, to complete a blood draw, a clinician may first couple a discard syringe to the luer connection port, draw a “waste blood” sample into the discard syringe, remove the discard syringe, couple a blood collection syringe to the connection port, draw a test blood sample into the blood collection syringe, remove the blood collection syringe, couple a saline flush syringe to the connection port, complete a saline flush, and remove the saline flush syringe. Such repeated connection and disconnection of the various syringes is inefficient and may increase the risk of patient infection due to contamination.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

In accordance with an aspect of the present disclosure, a blood collection system configured for connection to a tube set providing vascular access to a patient is disclosed. The blood collection system may include a discard syringe configured to collect a waste blood sample therein, the discard syringe including a primary connector portion and a secondary port, wherein the secondary port is in fluid communication with the primary connector portion, and a collection syringe configured to collect a testing blood sample therein, wherein the collection syringe includes a collection syringe connector portion configured to be couplable with the secondary port of the discard syringe.

In some embodiments, the secondary port is a self-sealing port.

In some embodiments, the secondary port is a needle-free access port.

In some embodiments, the secondary port is a split-septum port.

In some embodiments, the secondary port extends from a distal portion of the discard syringe.

In some embodiments, the discard syringe further includes a discard syringe body, a discard syringe plunger, and a discard syringe stopper, and the waste blood sample is drawn into the discard syringe by retraction of the discard syringe plunger and the discard syringe stopper.

In some embodiments, the collection syringe further includes a collection syringe body, a collection syringe plunger, and a collection syringe stopper.

In some embodiments, the testing blood sample is drawn into the collection syringe by retraction of the collection syringe plunger and the collection syringe stopper.

In some embodiments, the collection syringe is configured as an arterial blood gas (ABG) syringe.

In some embodiments, the blood collection system further includes a flushing solution syringe configured to contain a flushing solution therein.

In some embodiments, the flushing solution syringe is couplable to the secondary port of the discard syringe.

In some embodiments, the primary connector portion of the discard syringe is configured to be couplable to a luer connection port extending from a stopcock connector in fluid communication with the tube set.

According to another aspect of the present disclosure, a component packaging system for a blood collection system is provided. The component packaging system may include a tray having at least a first compartment, a second compartment, and a third compartment, wherein the first compartment is configured to hold a discard syringe, the second compartment is configured to hold a collection syringe, and the third compartment is configured to hold a flushing solution syringe. The system may also include a first flap pivotable with respect to the tray and configured to provide access to only the first compartment when moved from a first position of the first flap to a second position of the first flap, and a second flap pivotable with respect to the tray and configured to provide access to at least one of the second compartment and the third compartment when moved from a first position of the second flap to a second position of the second flap.

In some embodiments, the system may include a sterilizing swap coupled to a top surface of the first flap and accessible when the first flap is in the first position of the first flap.

In some embodiments, the system may include a peel-able film disposed over the first flap, the second flap, and an open portion of the tray.

In some embodiments, the tray further includes at least one storage recess, and the at least one storage recess is configured to hold a cap configured for use with the collection syringe.

In some embodiments, the at least the tray includes numerical indicia indicative of an order of use of components stored within the tray.

In accordance with another aspect of the present disclosure, a method of collecting a blood sample from a tube set providing vascular access to a patient is disclosed. The method may include providing a blood collection system, the blood collection system including a discard syringe configured to collect a waste blood sample therein, the discard syringe having a primary connector portion and a secondary port, wherein the secondary port is in fluid communication with the primary connector portion, and a collection syringe configured to collect a testing blood sample therein, wherein the collection syringe includes a collection syringe connector portion. The method may also include coupling the primary connector portion of the discard syringe to a connection port in fluid communication with the tube set, drawing a waste blood sample into the discard syringe, coupling the collection syringe connector portion to the secondary port of the discard syringe, and drawing a testing blood sample into the collection syringe.

In some embodiments, the method further includes providing a flushing solution syringe, decoupling the collection syringe from the secondary port after the testing blood sample is drawn into the collection syringe, coupling the flushing solution syringe to the secondary port of the discard syringe, and injecting a flushing solution contained within the flushing solution syringe into the patient.

In some embodiments, the method further includes injecting the waste blood sample held within the discard syringe into the patient before injecting the flushing solution contained within the flushing solution syringe into the patient.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a blood collection system in accordance with an aspect of the present disclosure;

FIG. 2A is a perspective view of a blood collection system of FIG. 1 in a first configuration;

FIG. 2B is a perspective view of the blood collection system of FIG. 1 in a second configuration;

FIG. 2C is a perspective view of the blood collection system of FIG. 1 in a third configuration;

FIG. 2D is a perspective view of the blood collection system of FIG. 1 in a fourth configuration;

FIG. 2E is a perspective view of the blood collection system of FIG. 1 in a fifth configuration;

FIG. 3 is a top view of component packaging for a blood collection system in accordance with an aspect of the present disclosure;

FIG. 4A is a perspective view of the component packaging of FIG. 3 in a first configuration;

FIG. 4B is a perspective view of the component packaging of FIG. 3 in a second configuration;

FIG. 4C is a perspective view of the component packaging of FIG. 3 in a third configuration;

FIG. 4D is a perspective view of the component packaging of FIG. 3 in a fourth configuration; and

FIG. 4E is a perspective view of the component packaging of FIG. 3 in a fifth configuration.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

In the present disclosure, the distal end of a component or of a device (e.g., a syringe) means the end furthest away from the hand of the user, and the proximal end means the end closest to the hand of the user, when the component or device is in the use position, i.e., when the user is holding a syringe in preparation or during use. Similarly, in this application, the terms “in the distal direction” and “distally” mean in the direction furthest from the hand of the user when in use, and the terms “in the proximal direction” and “proximally” mean in the direction closes to the hand of the user when in use.

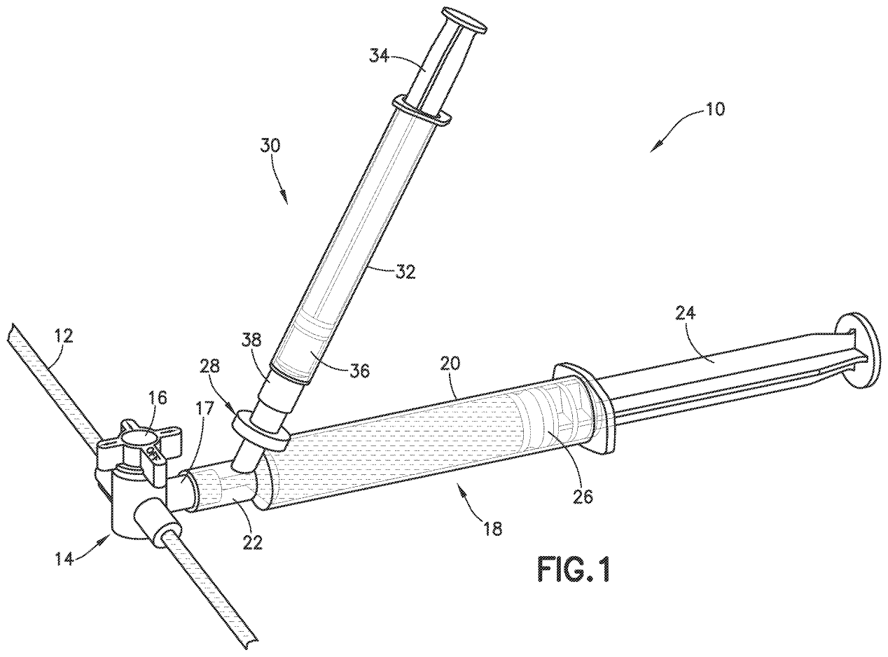

Referring to FIG. 1, a blood collection system 10 in accordance with an aspect of the present disclosure is illustrated. Blood collection system 10 includes a tube set 12 providing vascular access to the patient via, e.g., an A-line, a PIC, a catheter, etc. In the embodiment shown in FIG. 1, the tube set 12 is coupled to a stopcock connector 14, with stopcock connector 14 including a rotatable tap 16 configured to enable selective fluid passage between the various ports of the stopcock connector 14. A connection port 17 extends from one side of the stopcock connector 14, providing a single connection location for fluid communication with the tube set 12. In some embodiments, the connection port 17 may be a luer connection port.

In some embodiments, the blood collection system 10 includes a discard syringe 18, wherein discard syringe 18 is configured to be removably couplable to the connection port 17 of the stopcock connector 14 via a connector portion 22. The discard syringe 18 further includes a syringe body 20, a plunger 24, and a stopper 26. As will be described in further detail below, the discard syringe 18 is utilized to draw and store a waste blood sample from the patient prior to collection of testing blood sample.

Referring still to FIG. 1, the discard syringe 18 also includes a secondary port 28 extending from a distal end portion near the connector portion 22, with the secondary port 28 also being in fluid communication with the connector portion 22. In some embodiments, the secondary port 28 is configured as a self-sealing port such as, e.g., a BD Q-Syte™ connector from Becton, Dickinson and Co. The secondary port 28 may include a flat seal surface for improved disinfection of the connection point, and may provide needle-free access via a split septum configuration.

In some embodiments, the blood collection system 10 further includes a collection syringe 30. As will be described in further detail below, the collection syringe 30 may be utilized to collect a testing blood sample from the patient after a waste blood sample is drawn into the discard syringe 18. As shown in FIG. 1, the collection syringe 30 is couplable to the secondary port 28 of the discard syringe 18 via a connector portion 38. In some embodiments, the connector portion 38 is be configured as a male luer connector capable of accessing the self-sealing secondary port 28. Furthermore, in some embodiments, the collection syringe 30 includes a syringe body 32, a plunger 34, and a stopper 36. However, it is to be understood that collection syringe 30 may be any appropriate syringe or other container capable of collecting a blood sample therein.

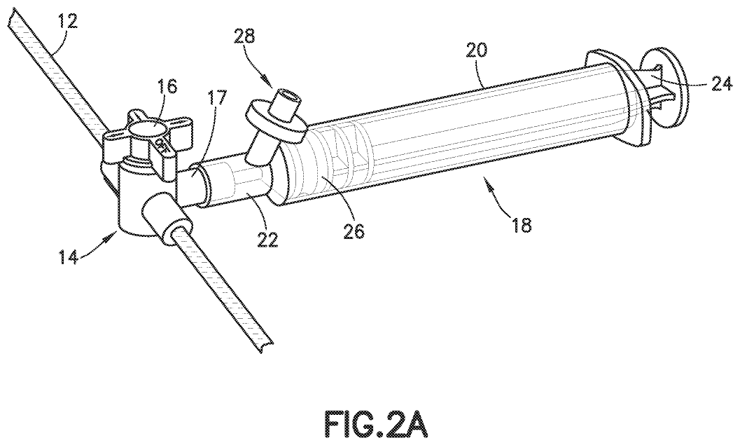

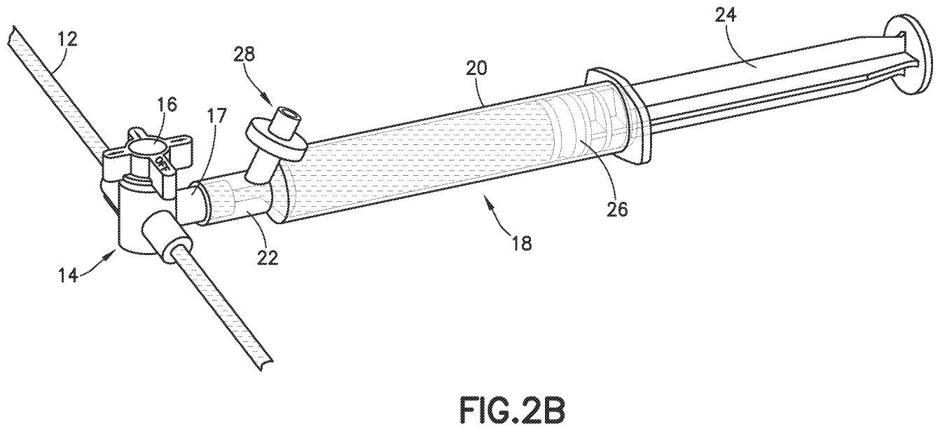

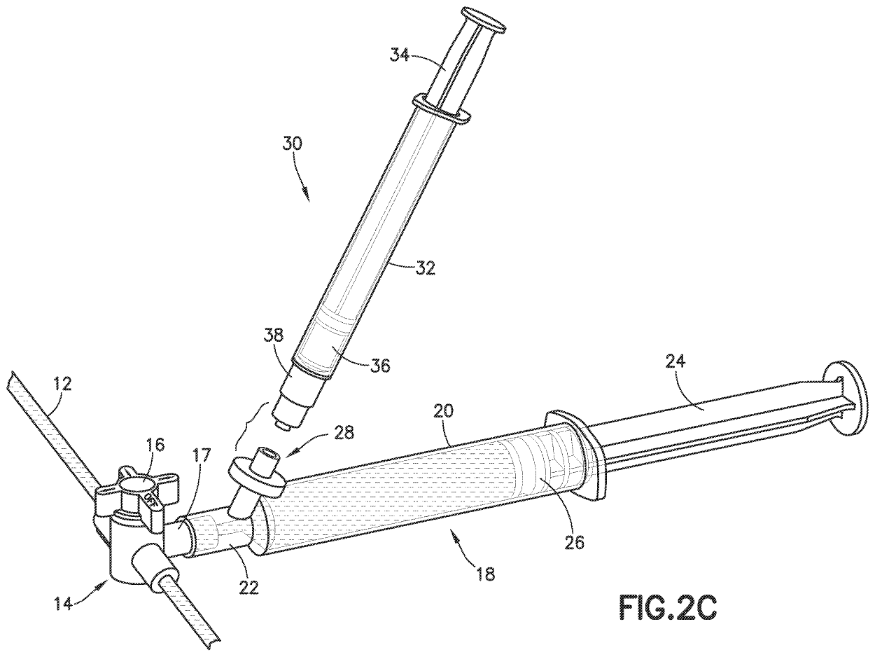

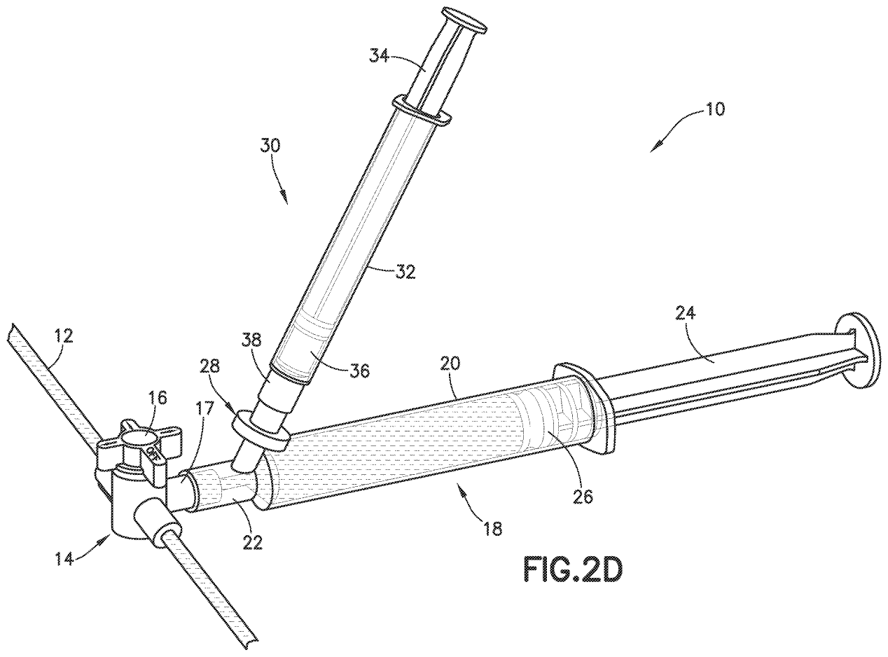

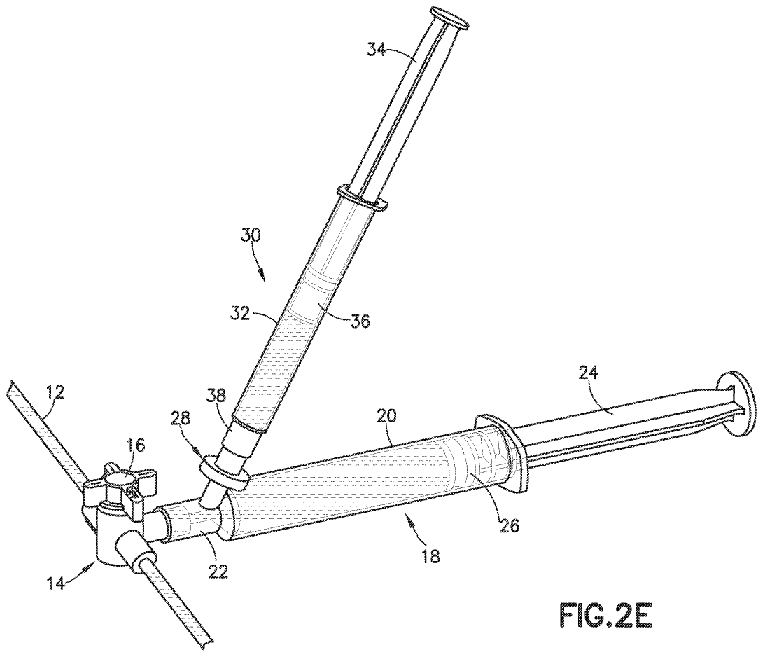

Referring now to FIGS. 2A-2E, various steps of use of the blood collection system 10 in accordance with an aspect of the present disclosure are illustrated. First, referring to FIG. 2A, after disinfection of the connection port 17 of the stopcock connector 14 with, e.g., an alcohol wipe or swab, the discard syringe 18 is coupled to the connection port 17. The clinician then rotates the tap 16 such that fluid communication between the connection port 17 and the tube set 12 is established, allowing the clinician to draw a waste blood sample into the discard syringe 18 by retraction of the plunger 24, as is shown in FIG. 2B.

Next, referring to FIGS. 2C and 2D, after a desired volume of waste blood is drawn in to the discard syringe 18, the clinician may couple the collection syringe 30 to the secondary port 28 of the discard syringe 18. As is shown in FIG. 2E, the clinician then draws a testing blood sample into the collection syringe 30. In some embodiments, the testing blood sample is drawn into the collection syringe 30 by manual withdrawal of the plunger 34, as is shown in FIG. 2E. However, in other embodiments, the collection syringe 30 may be configured to self-fill. For example, the collection syringe 30 may be configured as an arterial blood gas (ABG) vented syringe which, similar to an evacuated blood collection tube, may allow blood to be drawn into the collection syringe 30 via patient blood pressure alone.

While not shown in FIGS. 2A-2E, after collection of a desired testing blood sample into the collection syringe 30, the clinician may disconnect the collection syringe 30 from the secondary port 28. In some embodiments, the secondary port 28 is configured to automatically seal after removal of the collection syringe 30. The collection syringe 30 and the testing blood sample contained therein may then be managed per institutional guidelines. For example, the collection syringe 30 containing the testing blood sample may be transported and/or stored for future testing, or may utilized for immediate point-of-care testing.

After the collection syringe 30 is removed from the secondary port 28 and all desired blood draw procedures are completed, the clinician may couple a flushing solution syringe (not shown) to the secondary port 28 in order to flush the system with, e.g., a saline solution. In some embodiments and systems, the saline flush is initiated while the waste blood sample remains within the discard syringe 18. After the saline flush is completed, the clinician may rotate the tap 16 such that fluid communication between the connection port 17 and the tube set 12 is interrupted, and the discard syringe 18 can be decoupled from the connection port 17, allowing the waste blood sample to be discarded.

However, depending upon the type of system and institutional guidelines, in some embodiments, it may be desirable for the waste blood stored within the discard syringe 18 to be returned to the patient's system via the venous access device. In such embodiments, the waste blood is first injected back into the patient's vasculature by depression of the plunger 24 by the clinician. After infusion of the desired volume of waste blood is complete, the saline flush via a flushing solution syringe (not shown) coupled to the secondary port 28 is initiated. Upon completion of the saline flush, the discard syringe 18, which would no longer contains the waste blood sample, can be decoupled from the connection port 17 and discarded in an appropriate medical waste container.

With the blood collection system 10 described above with respect to FIGS. 1-2E, collection of a waste blood sample, a testing blood sample, and completion of a saline flush can be done via a single connection to the connection port 17 of the stopcock connector 14, thereby improving workflow efficiency for the clinician and reducing the risk of patient infection, as the system does not require repeated connection and disconnection of separate syringes to the connection port 17.

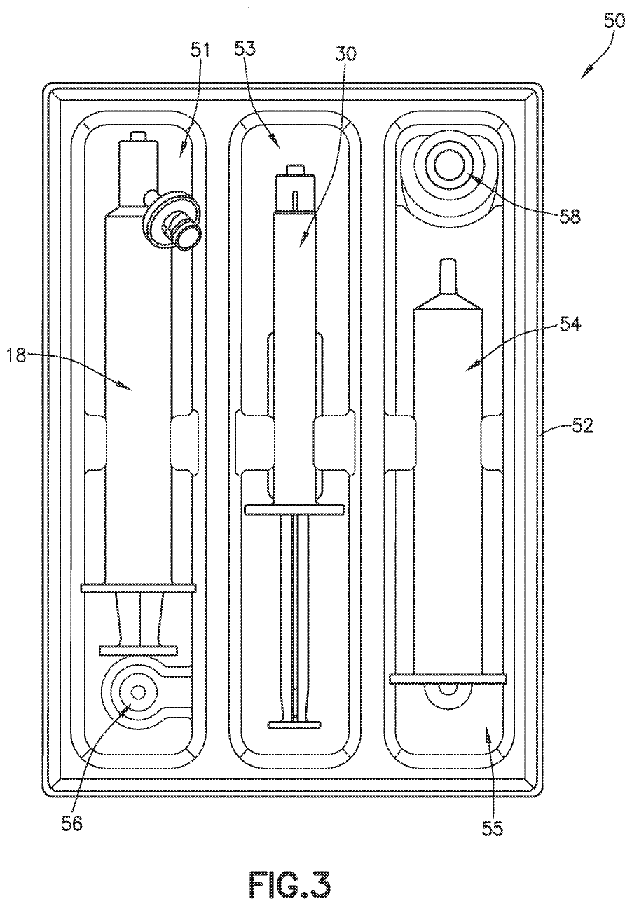

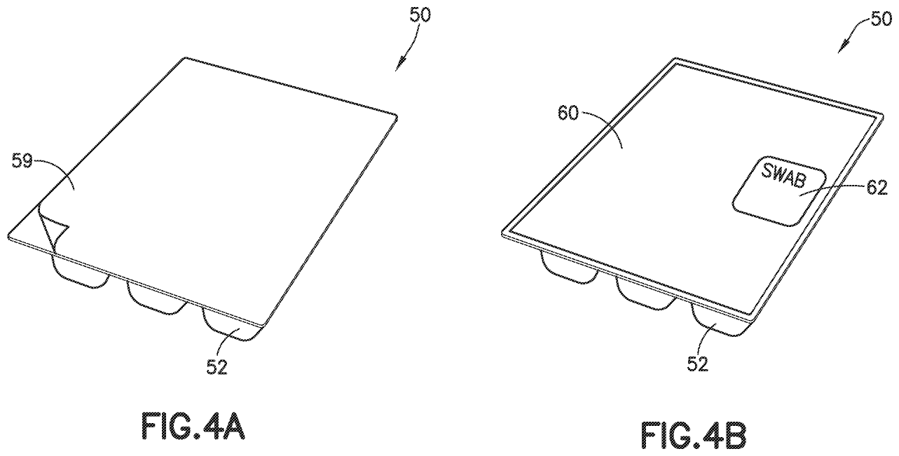

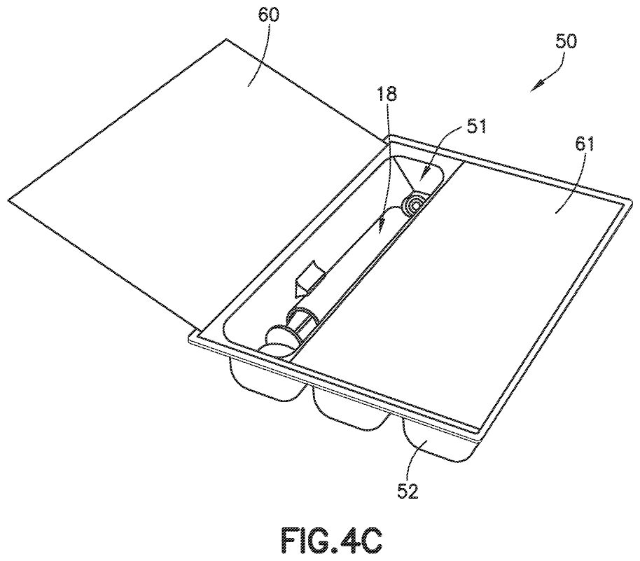

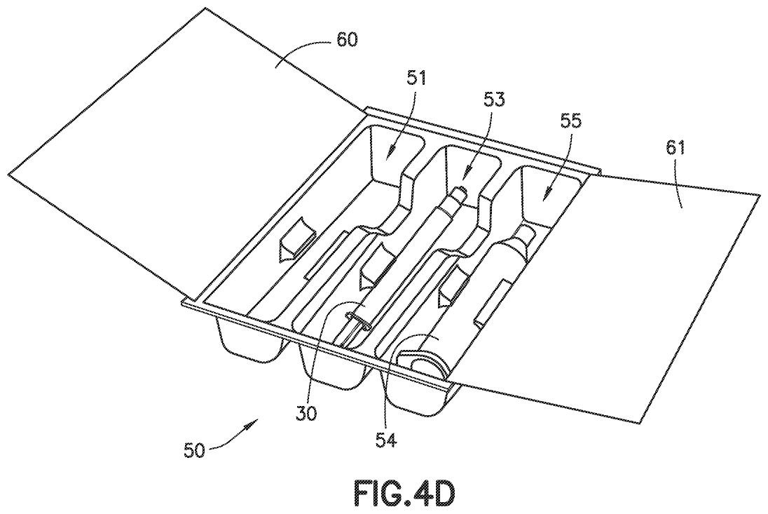

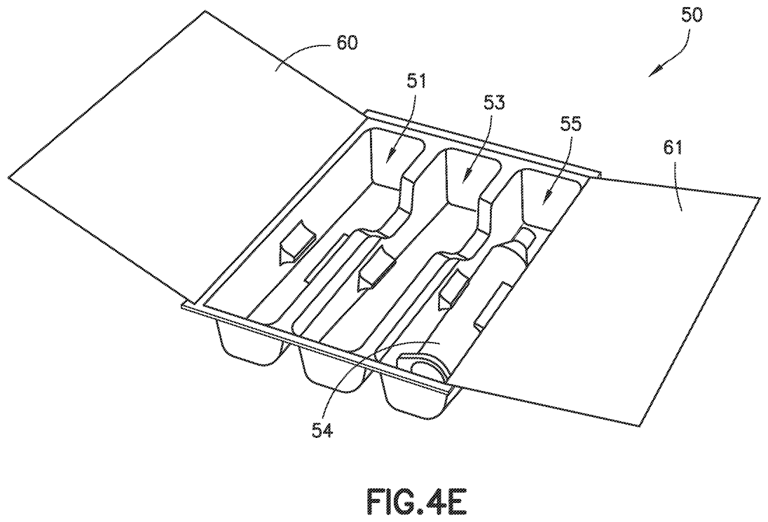

Referring now to FIGS. 3-4E, a component packaging system 50 for a blood collection system in accordance with an aspect of the present disclosure is illustrated. As will be described in further detail below, the component packaging system 50 may be configured to provide all or most of the necessary components for blood draw via a tube set coupled to an existing venous access device in a single package. Furthermore, the component packaging system 50 may be configured to guide the clinician toward use of the components contained therein in an appropriate order in accordance with institutional guidelines.

First, as shown in FIG. 3, the component packaging system 50 includes a tray 52 having a first compartment 51, a second compartment 53, and a third compartment 55. First compartment 51 is configured to hold the discard syringe 18, the second compartment 53 is configured to hold the collection syringe 30, and third compartment 55 is configured to hold a flushing solution syringe 54. In some embodiments, the flushing solution syringe 54 may be pre-filled with a flushing solution such as, e.g., a saline solution. The tray 52 may also include a first storage recess 56 and a second storage recess 58. In some embodiments, first storage recess 56 may be sized and configured to hold a hub cleaning cap (not shown) configured to clean, e.g., the secondary port 28 of the discard syringe 18 prior to connection of the collection syringe 30 to the secondary port 28. Furthermore, second storage recess 58 may be configured to hold a sample cap (not shown), which may be placed on the distal end of collection syringe 30 after a blood sample has been collected therein.

In some embodiments, some or all of the respective compartments 51, 53, 55 and recesses 56, 58 may include indicia such as numbering, thereby providing a guide to the clinician as to the correct order in which to attach and utilize the various components of the blood collection assembly. Furthermore, referring to FIGS. 4A-4E, the entire packaging assembly of component packaging system 50 may be configured such that the clinician is guided through the appropriate steps of a blood draw using the blood collection system described above.

As shown in FIG. 4A, the component packaging system 50 may be provided with a peel-able film 59 disposed on the tray 52, with the film 59 acting to maintain sterility of the various components in tray 52 during storage and prior to use. The film 59 may include written or other indicia so as to provide an indication to the clinician as to where and how to remove the film 59 and/or other instructions related to use of the components stored in the tray 52. Referring to FIG. 4B, once the film 59 is removed, a first flap 60 extending substantially over the tray 52 is exposed. In some embodiments, a sterilizing swab 62 (e.g., an alcohol swab) may be provided on an outer surface of the first flap 60, thus prompting the clinician to use the sterilizing swab 62 on, e.g., the connection port 17 of stopcock connector 14 before beginning the blood draw procedure.

Next, referring to FIG. 4C, the clinician opens the first flap 60, which exposes only the first compartment 51 holding the discard syringe 18, as a second flap 61 may be provided to extend over the second compartment 53 and the third compartment 55. In this way, the clinician is only able to access the discard syringe 18 upon initially opening the first flap 60, ensuring that the discard syringe 18 is the first component accessed prior to beginning the blood draw. After the discard syringe 18 is removed and coupled to the connection port 17, the clinician may then open the second flap 61, which provides access to both the second compartment 53 and third compartment 55, as is illustrated in FIGS. 4D and 4E. In this way, the clinician may be provided access to both the collection syringe 30 and the flushing solution syringe 54. In other embodiments, separate flaps may be provided to cover both second compartment 53 and third compartment 55, respectively.

Using the component packaging system 50 shown and described in FIGS. 3-4E, the clinician may be provided with all components necessary to conduct a blood collection procedure from a stopcock connector 14 such as that shown and described above with respect to FIGS. 1-2E. Additionally and/or alternatively, the component packaging system 50 may be configured such that the clinician is directed by indicia and/or selective component access to utilize the various components in the correct order, simplifying the blood draw process and aiding proper use of the blood collection system.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A blood collection system configured for connection to a tube set providing vascular access to a patient, the blood collection system comprising:
a discard syringe configured to collect a waste blood sample therein, the discard syringe comprising a primary connector portion and a secondary port, wherein the secondary port is in fluid communication with the primary connector portion; and
a collection syringe configured to collect a testing blood sample therein, wherein the collection syringe comprises a collection syringe connector portion configured to be couplable with the secondary port of the discard syringe.

2. The blood collection system of claim 1, wherein the secondary port is a self-sealing port.

3. The blood collection system of claim 1, wherein the secondary port is a needle-free access port.

4. The blood collection system of claim 3, wherein the secondary port is a split-septum port.

5. The blood collection system of claim 1, wherein the secondary port extends from a distal portion of the discard syringe.

6. The blood collection system of claim 1, wherein the discard syringe further comprises a discard syringe body, a discard syringe plunger, and a discard syringe stopper, and wherein the waste blood sample is drawn into the discard syringe by retraction of the discard syringe plunger and the discard syringe stopper.

7. The blood collection system of claim 1, wherein the collection syringe further comprises a collection syringe body, a collection syringe plunger, and a collection syringe stopper.

8. The blood collection system of claim 7, wherein the testing blood sample is drawn into the collection syringe by retraction of the collection syringe plunger and the collection syringe stopper.

9. The blood collection system of claim 7, wherein the collection syringe is configured as an arterial blood gas (ABG) syringe.

10. The blood collection system of claim 1, further comprising a flushing solution syringe configured to contain a flushing solution therein.

11. The blood collection system of claim 10, wherein the flushing solution syringe is couplable to the secondary port of the discard syringe.

12. The blood collection system of claim 1, wherein the primary connector portion of the discard syringe is configured to be couplable to a luer connection port extending from a stopcock connector in fluid communication with the tube set.

13. A component packaging system for a blood collection system comprising:
a tray having at least a first compartment, a second compartment, and a third compartment, wherein the first compartment is configured to hold a discard syringe, the second compartment is configured to hold a collection syringe, and the third compartment is configured to hold a flushing solution syringe;
a first flap pivotable with respect to the tray and configured to provide access to only the first compartment when moved from a first position of the first flap to a second position of the first flap; and
a second flap pivotable with respect to the tray and configured to provide access to at least one of the second compartment and the third compartment when moved from a first position of the second flap to a second position of the second flap.

14. The component packaging system of claim 13, further comprising a sterilizing swap coupled to a top surface of the first flap and accessible when the first flap is in the first position of the first flap.

15. The component packaging system of claim 13, further comprising a peel-able film disposed over the first flap, the second flap, and an open portion of the tray.

16. The component packaging system of claim 13, wherein the tray further comprises at least one storage recess, and wherein the at least one storage recess is configured to hold a cap configured for use with the collection syringe.

17. The component packaging system of claim 13, wherein at least the tray comprises numerical indicia indicative of an order of use of components stored within the tray.

18. A method of collecting a blood sample from a tube set providing vascular access to a patient, the method comprising:
providing a blood collection system, the blood collection system comprising:
a discard syringe configured to collect a waste blood sample therein, the discard syringe comprising a primary connector portion and a secondary port, wherein the secondary port is in fluid communication with the primary connector portion, and
a collection syringe configured to collect a testing blood sample therein, wherein the collection syringe comprises a collection syringe connector portion;
coupling the primary connector portion of the discard syringe to a connection port in fluid communication with the tube set;
drawing a waste blood sample into the discard syringe;
coupling the collection syringe connector portion to the secondary port of the discard syringe; and
drawing a testing blood sample into the collection syringe.

19. The method of claim 18, further comprising:
providing a flushing solution syringe;
decoupling the collection syringe from the secondary port after the testing blood sample is drawn into the collection syringe;
coupling the flushing solution syringe to the secondary port of the discard syringe; and
injecting a flushing solution contained within the flushing solution syringe into the patient.

20. The method of claim 19, further comprising injecting the waste blood sample held within the discard syringe into the patient before injecting the flushing solution contained within the flushing solution syringe into the patient.
